(19) United States
(12) Patent Application Publication
(10) Pub. No.: US 2013/0190608 A1
(43) Pub. Date: Jul. 25, 2013

(54) ORAL COIL FOR A MAGNETIC RESONANCE TOMOGRAPHY SYSTEM

(71) Applicant: Sebastian Schmidt, Weisendorf (DE)

(72) Inventor: Sebastian Schmidt, Weisendorf (DE)

(73) Assignee: Siemens Aktiengesellschaft, Munchen (DE)

(21) Appl. No.: 13/554,947

(22) Filed: Jul. 20, 2012

(30) Foreign Application Priority Data
Jul. 21, 2011 (DE) 10 2011 079 577.4

(51) Int. Cl. A61B 5/055

(52) U.S. Cl. CPC A61B 5/055
USPC 600/422

(57) ABSTRACT

An oral coil for a magnetic resonance tomography system for creation of magnetic resonance images of a jaw area of a patient includes a high-frequency antenna system with a number of high-frequency antennas and a shim system. The shim system includes a number of shim elements for homogenization of a static basic magnetic field of the magnetic resonance tomography system.

This application claims the benefit of DE 10 2011 079 577.4, filed on Jul. 21, 2011.

### BACKGROUND

The present embodiments relate to an oral coil for a magnetic resonance tomography system for creating magnetic resonance images of a jaw area of a patient.

Diseases of the teeth and of the periodontal apparatus (e.g., periodontosis or karies) may be diagnosed with x-ray-based imaging methods. X-ray technologies used for this purpose extend from conventional x-ray methods through digital x-ray methods in projection mode to innovative 3D x-ray methods. In 3D x-ray diagnostics, for example, there are digital volume diagnostic technology (DVT) systems on the market, in which complete areas of the jaw are imaged, or in addition, high-resolution 3D images of teeth and jaw areas may be prepared. The radiation of the digital system is reduced by comparison with conventional diagnostics, and an image is available immediately. DVT systems allow a type of x-ray computer tomography of the teeth and of the facial portion of the skull with high resolution and positional accuracy. Such DVT diagnostic systems are, however, very complex and expensive, so that 3D x-ray diagnostics is only used with a few indications. Therefore, there is a need for new imaging systems and methods in the jaw area.

For many diseases of the teeth or of the periodontal apparatus, a magnetic resonance tomography (MRT) examination may be a good alternative to the previous methods (e.g., x-ray diagnostics), since MRT examination is free from ionizing radiation and also makes better presentation of soft tissue contrasts possible. The systems used for this however are either too expensive or have an image quality that is too low.

In a magnetic resonance device, the body to be examined may be subjected with the aid of the basic magnetic field system to a defined basic magnetic field (e.g., the B₀ field). In addition, a magnetic field gradient is applied with the aid of a gradient system. High-frequency magnetic resonance excitation signals (HF signals) with a defined field strength are sent out by suitable antennas via a high-frequency transmission system. The magnetic flux density may be designated B₁. The pulse-type high frequency field may therefore also be called the B₁ field for short. Using these high frequency pulses, the nuclear spin of specific atoms excited resonantly by this high frequency field are flipped by a defined flip angle in relation to the magnetic field lines of the basic magnetic field (B₀ field). During the relaxation of the nuclear spin, high-frequency signals (e.g., magnetic resonance signals) are again emitted. The emitted high-frequency signals are received and further processed by suitable receive antennas. The desired magnetic resonance image data (MR image data) may be reconstructed from the magnetic resonance signals or “raw data” thus received. Local encoding is undertaken by switching appropriate magnetic field gradients in the different spatial directions at precisely defined times (e.g., during the sending out of the HF signals and/or during receipt of the magnetic resonance signals). The high-frequency signals for nuclear spin magnetization may be sent out by a “body coil” permanently installed in the magnetic resonance tomograph. A typical layout for this is a birdcage antenna that includes a number of transmit rods that are arranged running in parallel to the longitudinal axis around a patient chamber of the tomograph, in which an object under examination (e.g., a patient) is located during the examination. On end face sides, the antenna rods are each connected to one another in the shape of a ring. As an alternative to the body coils (e.g., body scanners), for examining areas of the patient, magnetic resonance systems with a very much smaller field of view (FOV), in which significantly lower expenditure on apparatus for small subareas delivers very good image quality, are used. Such systems may be used in the area of jaw imaging. The dedicated jaw or head scanners are more sensible here than whole-body scanners because of the overall size of the magnetic resonance system and because of the homogeneity volume needed, where the dedicated jaw or head scanners deliver a comparable image quality.

To receive the magnetic resonance signals, local coils with a high signal-to-noise ratio are used. These are antenna systems that are attached in the immediate vicinity of the patient. The magnetic resonance signals induce a voltage in the individual antennas of the local coils. The induced voltage is amplified with a low-noise preamplifier (LNA, Preamp) and forwarded over a cable to the receive electronics. To improve the signal-to-noise ratio (e.g., with high-resolution images), high-field systems are used. These systems operate with a basic magnetic field B₀ of 1.5 to 12 Tesla and more.

Of importance with many magnetic resonance applications (e.g., clinical MRT) is the homogeneity of the B₀ basic magnetic field. Artifacts or distortions may thus arise with deviations in the homogeneity, for example. A distortion of the B₀ basic magnetic field may also occur in different regions of the head. The reason for this is a spatially strongly inhomogeneous distribution of the susceptibility of the head tissue. In this case, the susceptibility (e.g., specified as the magnetic volume susceptibility χ_{V}) is a measure for the magnetizability of material in an external magnetic field and has a simple relationship to the magnetic permeability μᵣ (e.g., μᵣ=χ_{V}+1).

An additional complicating factor is that most local coils for MRT diagnostics of the head area are built so that with head examinations, the forehead or the center of the head lies in the isocenter of the MRT system. This is relevant with dedicated head coils with a small FOV. In MRT diagnostics of the jaw area with such head coils, seen from the center of the head, the jaw area lies ventrally (e.g., seen from the central axis of the head) offset in the direction of the front side of the head or towards the face. This ventral location of the jaw provides that in the case of a jaw area image, the imaging volume with a normal head coil is, however, off-center and thus does not lie in the optimum homogeneity volume of the B₀ field.

The distortions occurring through the inhomogeneity of the B₀ field may be corrected by shim coils that, like the body coil, are permanently built into magnetic resonance tomographs. The number of different shim coils in magnetic resonance tomographs, the arrangement of the shim coils, and the control of the shim coils have a restricted number of degrees of freedom, however, in order to compensate for a B₀ inhomogenity of the mostly superconducting basic magnetic field system by shim currents in conventional copper coils. The number of degrees of freedom is thus not sufficient in many conventional MR systems, because of the ventral location of the area to be examined, to enable an inhomogeneity of the B₀ field to be sufficiently compensated for.

As an alternative to the shim coils built into the MR system (e.g., if the orders of shim coils integrated into the device or degrees of freedom are not sufficient), attempts have been made to compensate for the inhomogeneity in the B₀ field when recording images of teeth, for example, by filling the mouth cavity of the patient with water. This is intended, for example, to improve the shim as a result of compensating for the local susceptibility differences in the mouth cavity. The filling of the mouth cavity with water may however, as a result of the position of the jaw during the measurement, not compensate for subtle differences in a differentiated manner and is very uncomfortable for the patient.

### SUMMARY AND DESCRIPTION

The present embodiments may obviate one or more of the drawbacks or limitations in the related art. For example, an improved alternative to the previous local coils for the jaw area (e.g., oral coils) and methods for creating improved magnetic resonance images with the oral coil are provided.

An oral coil for a magnetic resonance tomography system includes a high-frequency antenna system with a number of high-frequency antennas and a shim system with a number of shim elements for homogenization of a static basic magnetic field of the magnetic resonance tomography system. The static basic magnetic field applied from outside by the basic magnets is the B₀ field.

Oral coils are local coils for magnetic resonance tomography of the jaw area. For example, an oral coil is a coil that is adapted for a magnetic resonance system with a small field of view (FOV). Such magnetic resonance systems are, for example, dedicated head scanners with a small homogeneity volume that is sufficient for the jaw area. Head scanners may include jaw scanners. Such dedicated head scanners may be equipped at very much lower cost compared to body scanner systems, since the head scanners do not have to create such a large homogeneity volume. The relocation or moving of the patient in the magnetic resonance system is not possible or is only possible to a limited extent compared to whole-body scanners, so that without the oral coil of the present embodiments, the regions of interest for imaging may lie outside the FOV or at an edge of the FOV.

Images of the jaw area are images that are made in a spatially restricted area of an area of the jaw of the patient (e.g., the upper jaw area or the lower jaw area, the entire jaw or the jaw joint, either alone or together with further jaw areas and/or a number of teeth or the periodontal apparatus, by contrast with complete head, whole-body or overview imaging that also includes areas of the jaw among other areas). Such oral coils may have poor signal-to-noise ratio and may not deliver adequate image quality. In such cases, one or more teeth that may both be adjacent to one another but also lie in different jaw areas (e.g., in the lower and upper jaw) may be the object of the examination. A patient for whom the magnetic resonance images of the jaw area are to be produced may be a human or an animal.

As mentioned, the jaw areas, because of the ventral location of the jaw of the patient, are disposed outside or at the edge of the FOV (e.g., ventrally when seen from the isocenter of the overall measurement system). Integrating a shim system with a number of shim elements into an oral coil is advantageous, since a homogenization of the static basic magnetic field may be achieved with simple devices. By using the shim system with a number of shim elements directly in or on the oral coil, an improvement of the signal-to-noise ratio and an improved reproducibility may be achieved without having to make the overall system any larger.

It is in the jaw area, for example, that the usual air-filled mouth cavity and teeth fillings, crowns or implants, for example, affect the homogeneity of the magnetic field greatly, so that the result is local changes and shifts of the magnetic field lines of the basic magnetic field. The oral coil of the present embodiments makes it possible to compensate for an inhomogeneity of the magnetic field that is based on inhomogeneous tissue structures or materials in the mouth or in the jaw.

One embodiment of a high-frequency receive device for a magnetic resonance tomography system for creating magnetic resonance images of the jaw area thus has at least one oral coil that is connected to a receive unit of the high-frequency receive device (e.g., a normal receive channel for connecting a local coil). The receiver unit further processes the signal detected with the local coil and digitizes the detected signal, for example. One embodiment of the magnetic resonance system for creating magnetic resonance images of the jaw area includes a head scanner with a basic field magnet 11 that has a smaller FOV (e.g., a dedicated head scanner). Head scanners may include jaw scanners. In addition to the usual components known to the person skilled in the art (e.g., the basic field magnet system and a gradient system), the magnetic resonance system also includes one embodiment of a high-frequency receive device or oral coil.

The oral coil is adapted to such a dedicated head scanner. Such scanners may have a FOV that is comparable to that of a typical head scanner (e.g., a spherical FOV between around 15 cm and around 30 cm in diameter). As an alternative to a spherical FOV, the scanner may also have a cylindrical FOV. In one embodiment, the FOV may have a homogeneity volume of around 10 cm in length (e.g., in the z-direction of the B₀ field—also the longitudinal axis of the patient) and around 25 cm in the x- or y-direction (e.g., perpendicular to the z-direction). Thus, in these areas, a very high homogeneity is achievable in a simple manner and also at relatively low cost with a low outlay in terms of apparatus. This homogeneity volume, however, is partly too small for a complete image of the jaw area. For use of the oral coil in such a dedicated jaw scanner, this homogeneity volume that is actually too small may be expanded such that a sensible imaging (e.g., of the front teeth) of the overall jaw area may still be achieved.

The oral coil may not be used to expand the homogeneity volume but may be used to change the homogeneity volume (e.g., deform the homogeneity volume), so that, for example, on a rear side (e.g., in an area of the nape of the neck of the patient), a homogeneous field is no longer present, but the homogeneity is expanded or improved in the jaw area. Since in the examination of the jaw area only the jaw or the teeth are of interest, a deterioration of the imaging in the area of the nape of the neck is of no significance. Therefore, the imaging by the oral coil is improved in the area of interest for the dental surgeon or jaw surgeon.

In one embodiment of a method for creating magnetic resonance images of a jaw area of the patient during an acquisition of magnetic resonance signals (e.g., using a dedicated head scanner) using a number of high-frequency antennas of the high-frequency antenna system of an oral coil for homogenization of the static basic magnetic field of the magnetic resonance tomography system, a shim system with a number of shim elements integrated into the oral coil is used.

This use of an oral coil that, as well as the high-frequency antenna system with a number of high-frequency antennas, has a shim system with a number of shim elements for homogenization of the static basic magnetic field of the magnetic resonance tomography system, enables the disadvantages of the conventional methods to be overcome. For example, an improvement in the reproducibility and the signal-to-noise ratio is achievable in this way.

Further advantageous embodiments and developments of the oral coil, of the high-frequency receive device, of the magnetic resonance system and of the method or the use of the method are described below. The oral coil, the high-frequency receive system, the magnetic resonance tomography system, the method and the use of the method may also be embodied in accordance with the other categories.

The high-frequency antenna system and the shim system form a constructional unit in one embodiment of an oral coil. A constructional unit may be that the high-frequency antenna system and the shim system may be embodied as a common part. As an alternative, the high-frequency antenna system and the shim system may be formed from different units and may be connected to each other in a removable manner. One option in this case is for the shim system to be pushed onto the high-frequency antenna system by push-fit connections. As an alternative, the shim system may be installed behind the high-frequency antenna system via a corresponding connection device such as plug-in connectors, for example, with corresponding holder systems providable for a fixed installation position during the measurement.

In the variants, the shim system may be attached to the high-frequency antenna system (e.g., seen from the mouth), behind the high-frequency antenna system. A reversed arrangement (e.g., with a shim system disposed closer to the object under examination) may also be provided even if, for simplification, only the first alternative is discussed in greater detail below.

In one embodiment, the oral coil may include a curved housing for use inside or in front of the mouth, essentially following the curve of the jaw, for the high-frequency antenna system and/or the shim system. This provides that the housing is adapted or may be adapted in a curved shape to the essentially parallel cross-sectional plane running between the upper and lower jaw of a patient.

In one embodiment, the housing may be disposed at a distance from the mouth or from the rows of teeth that essentially remains the same. The housing of the oral coil extends in, for example, from the right ear to the left ear of the patient in a largely symmetrical embodiment, so that the housing covers the entire jaw area. In another embodiment, the housing may also cover the left or right side of the jaw. The oral coil may be embodied so that the oral coil may be used both for the left and for the right jaw area side. Left and right may be that the corresponding directions are specified looking down from above onto the back of a patient's head (e.g., in the direction of view of the patient).

In one embodiment, the high-frequency antenna system and the shim system may be disposed in such a housing. The respective system may be disposed permanently integrated into the housing, or the housing may have corresponding receptacles for the respective system.

The shim system permanently integrated into the housing or disposed in a housing of the shim system may include holders for shim elements. The holders may be embodied so that the holders accommodate individual shim elements or alternatively a number of shim elements. A holder for a shim element may be a device, into which a shim element may be built temporarily (e.g., only during operation of the oral coil) or permanently. Holders for shim elements depend on the size and the shape of the shim elements and are embodied accordingly. For example, the holders may be embodied in the form of latching elements, into which individual elements or a number of shim elements may be inserted. The latching elements may be held by a latching mechanism in the holder. As an alternative, holders in the form of chambers or the like may also be used. A number of shim elements are inserted into the chambers. Depending on the shape of the shim elements, a number of shim elements (e.g., in a row arrangement) may also be provided in one holder. The advantage of this is that shim-active elements may also alternate with inactive elements, or shim elements with different shim strengths may be specifically selected and positioned relatively simply depending on requirements for the shim.

The holders may be configured so that an array of shim elements may be realized during operation. The term array may be that the shim elements may be disposed in rows and/or columns or with defined spacings between the individual rows and columns. In such cases, for example, an almost regular array of shim elements may be constructed.

Depending on the shape and design of the shim elements, this enables arrays to be constructed from shim elements, in which, depending on the occupation of the individual rows and columns by shim elements, a tailoring of the shim to the current requirements may be achieved. Since the B₀ field inhomogeneity is different depending on the object under examination and the location of the examination area in relation to the isocenter of the measurement system, this enables an individual adaptation to be achieved. The image quality, for example, may be improved by this capability of individual adaptation to the respective examination area.

In one embodiment of the oral coil, at least one of the shim elements may be a passive element made from a material with defined magnetic susceptibility for adaptation of a B₀ field inhomogeneity. The material used may have a magnetic susceptibility χ at room temperature of greater than 10² (e.g., greater than 10³).

Examples of such materials that have the given susceptibilities are diamagnetic materials and may be materials that include iron, cobalt, nickel or alloys of these metals, (e.g., iron and iron alloys).

In one embodiment of the oral coil, at least one of the shim elements may be an electrical shim coil. In one embodiment, such an electrical shim coil is embodied for local modification of a B₀ field inhomogeneity. A number of the shim elements (e.g., three, four, five, six or seven) may be electrical shim coils. The electrical shim coils, together with passive shim elements, may be used in combination.

In one embodiment of the oral coil, the shim system may be a shim coil array including a number of shim coils. Shim coil array may be that a number of shim coils disposed in rows and/or columns are integrated into the housing of the shim system. In such cases, the array may include just one row or also two, three or more rows disposed largely in parallel. Correspondingly, a number of one, two, three or even more columns is used. In another embodiment, a number of shim coils (e.g., five, six or seven shim coils) are present in the housing of the oral coil, disposed adjacently in a row in a curved area along the jaw to enable the entire jaw area to be covered, for example.

By a further reduction of the distance of the high-frequency antenna system from the examination area, a further improvement of the signal-to-noise ratio may be achieved. The high-frequency antenna system may be assigned at least partly in a housing part disposed intraorally during operation. This is of advantage for the examination of individual teeth or smaller tooth/jaw areas.

As an alternative or in addition, the high-frequency antenna system, or at least a part thereof, may be disposed in a housing part that is located during operation outside the patient's head. The combination enables improved positioning among other advantages, since the receipt of the high-frequency radiation may be measured from a number of angles of view.

An embodiment of the oral coil includes the shim elements in a housing part that is disposed during operation outside the patient's head. This enables a high shim accuracy to be achieved without having to make additional space available within the mouth cavity for the shim system. This embodiment also provides improved comfort for the patient since the equipment disposed within the mouth space may be designed as small as possible or not to be necessary at all.

### BRIEF DESCRIPTION OF THE DRAWINGS

The same components are provided with identical reference characters in the various figures.

FIG. 1 shows a schematic cross section through a first exemplary embodiment of an oral coil;

FIG. 2 shows a schematic view from the side of one embodiment of the oral coil in accordance with FIG. 1;

FIG. 3 shows a schematic longitudinal section through an oral coil in accordance with a second embodiment; and

FIG. 4 shows a schematic cross section through one embodiment of the oral coil in accordance with FIG. 3.

### DETAILED DESCRIPTION OF THE DRAWINGS

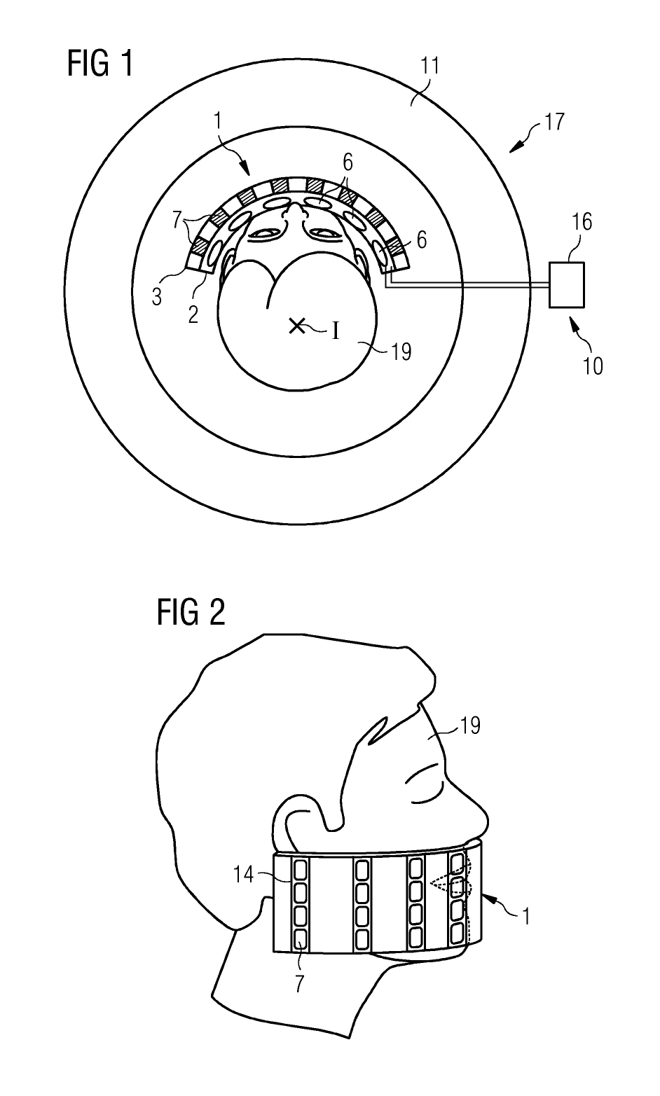

FIG. 1 shows an oral coil 1 in accordance with a first embodiment and use of the oral coil 1 as a part of a high-frequency receive device 10 of a magnetic resonance tomography system 17. Of the magnetic resonance tomography system, only the basic field magnet of the dedicated head coil that extends in a ring around a patient space, in which a patient 19 or a part of the patient (e.g., the head of the patient) is supported, is illustrated in FIG. 1 by way of example in addition to the high-frequency receive device formed by the oral coil 1 and a receive unit 16. The patient 19 is supported so that the oral coil 1 is disposed ventrally to an isocenter I that may be defined in the center of the basic field magnet 11 in the magnetic resonance tomography system 20. The magnetic resonance tomography system 17 has all the usual components known to the person skilled in the art (e.g., a gradient system, a body coil and if necessary, further local coils). These components are, however, not shown in FIG. 1 in order to simplify the diagram.

The oral coil 1 includes a high-frequency antenna system 2 with a number of high-frequency antennas 6, and a shim system 3 with a number of passive shim elements 7. The high-frequency antennas 6 of the oral coil 1 are connected to a receive unit 16 that further processes (e.g., amplifies, filters, and/or mixes to the appropriate frequency) and digitizes the magnetic resonance signals received from the high-frequency antennas 6.

Both the high-frequency antenna system 2 and the shim system 3 are disposed in a curved housing, in which the high-frequency antennas 6 are disposed in rows almost equidistantly from the mouth or jaw area of the patient 19. FIG. 1 shows a sectional view (in a sectional plane in parallel between the upper and lower jaw) for an embodiment, in which a row of six high-frequency antennas 6 disposed next to one another is integrated into the curved housing. A number of such rows may be disposed one above the other. The curved housing of the high-frequency antenna system 2 extends in this case roughly from the left ear to the right ear of the patient 19.

Like the high-frequency antenna system 2, the shim system 3 is also embodied from a curved housing with an almost equal bending radius. In the embodiment shown in FIG. 1, and viewed from the patient, the shim system 3 lies behind the high-frequency antenna system 2 (e.g., further outwards). The shim system 3 includes a number of columns of passive shim elements 7 that are disposed in the curved housing with a defined spacing in relation to one another in a row. In the section shown in FIG. 1, eight columns with passive shim elements 7 that are inserted into corresponding folders or pockets (not shown) are shown. In this case, as can be seen from FIG. 2, a number of rows of such shim elements are disposed above one another in order to improve the shim. Use of a number of rows and columns of individual shim elements 7, through suitable selection of the strength of the magnetic susceptibility of the individual shim elements 7, enables a more individual adjustment of the shims to be achieved and thus the inhomogeneity compensated for.

As an alternative to the arrangement of the shim elements 7 shown in FIG. 1, the elements may sit closely alongside and above one another (e.g., a full surface attachment of shim elements is present). This enables an even finer tuning of the shim to be achieved.

FIG. 2 shows a view from the side of the oral coil 1 of the first embodiment. FIG. 2 shows how the oral coil 1 is disposed around the mouth of the patient 19 in the area of the jaw (e.g., in the vertical alignment from roughly the nose to roughly the chin of the patient 19). The oral coil 1 has holders 14 for passive shim elements 7 in the area of the shim system 3.

In this case, the holders 14, as is shown in FIG. 2, may be lengthwise recesses or pockets, into which one or more shim elements 7 may be pushed. The holders 14 may in such cases extend vertically over almost the entire area of the oral coil. As shown in FIG. 2, a number of holders 14 may be disposed in a horizontal direction largely parallel to one another, so that a shim element array with a number of columns and rows is produced.

The shim elements 7 pushed into the holders are passive shim elements that include iron as the diamagnetic material. Depending on the size of the deviation of the B₀ field from the ideal homogeneity status, each shim element may include different iron content in order, by the respective magnetic susceptibility of the shim elements 7, to adjust the B₀ field accordingly. Through this, the homogeneity may be improved. The choice of the shim elements with suitable susceptibility depends on the corresponding strength of the inhomogeneity. If there is no inhomogeneity present at a specific point (e.g., because the distance from the isocenter is not so great), an inactive shim element 7 may also be inserted at the corresponding location of the shim system 3. For a large inhomogeneity, a shim element with a larger magnetic susceptibility (e.g., with a high iron content) may be inserted in the immediate vicinity of the inhomogeneity in order to compensate appropriately.

The fact that the shim elements 7 are easily accessible provides that the shim system may be adjusted in a simple manner (e.g., by replacing individual shim elements 7).

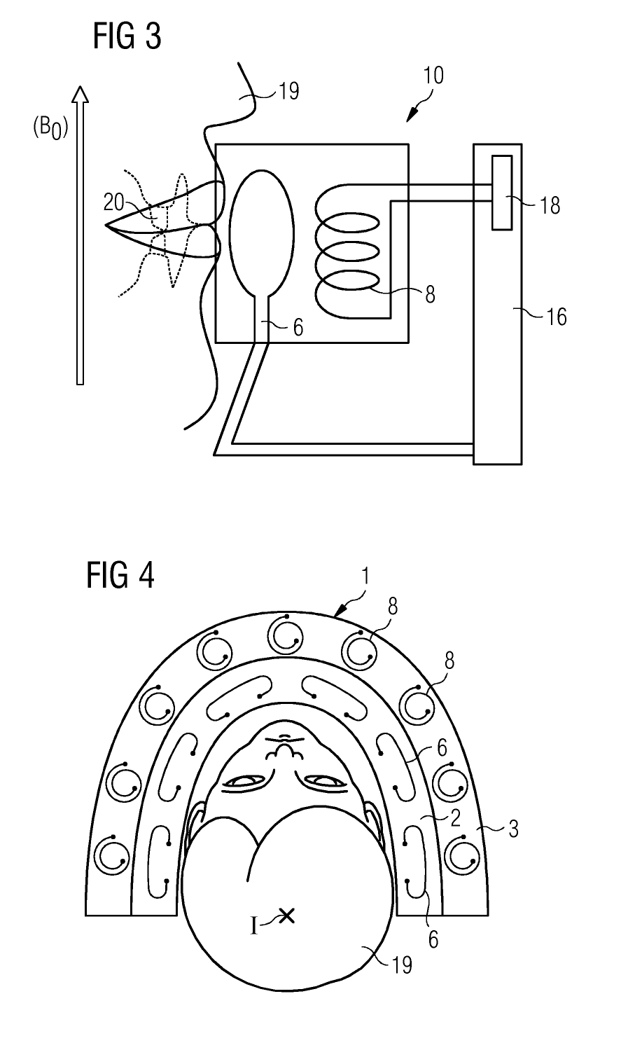

FIGS. 3 and 4 show a second embodiment of an oral coil 1, in which both high-frequency antennas 6 and electrical shim coils 8 are integrated into one housing. The high-frequency antennas 6 of the oral coil 1 are also connected, for example, to a receive unit 16 that further processes the magnetic resonance signals. The shim coils 8 are connected to a shim control unit 18 (e.g., integrated into the receive unit 16) for controlling the electrical shim coils 8. This may, for example, be controlled by a measurement controller (not shown) that, on the basis of adjustment measurements, establishes whether and where the basic magnetic field has an inhomogeneity and accordingly outputs control signals to the shim control unit 18. The shim control unit 18 applies suitable current of the appropriate polarity to the shim coils 8, so that the desired local magnetic field is embodied around the relevant shim coil 8 for homogenization of the basic magnetic field in this area. The inhomogeneity in the B₀ field may in such cases be compensated for by fine tuning. For this purpose, the shim coils 8 may be individually activated via separate shim control units 18. As an alternative, a number of electrical shim coils may also be activated by one shim control unit 18.

The oral coil is disposed outside the mouth of the patient 19 at the height of the teeth 20. In a similar way to the first embodiment, the high-frequency antenna system with the high-frequency antennas 6 is disposed in the housing of the oral coil closer to the shim system with the shim coils 8 in order to avoid, where possible, a disruption by the shim coils 8 of the high-frequency signals received by the high-frequency antennas 6.

FIG. 4 shows a cross-section (similar to that shown in FIG. 1 for the first exemplary embodiment) of one embodiment of an oral coil, from which a central location of the examination area of the patient 19 and thus the oral coil 1 in relation to the isocenter I is clearly visible.

From this overhead view, the curved embodiment of the housing of the oral coil with the high-frequency antenna system 2 and the shim system 3 is shown. Disposed in a row (e.g., lying next to one another) in the high-frequency antenna system 2 are a number of (e.g., six) high-frequency antennas. The shim system 3, further away when viewed from the patient outwards, includes a number of (e.g., nine) shim coils 8 disposed next to one another, with further rows of shim coils being disposed below or above the row in each case.

The oral coils, the high-frequency receive device and the method of use thereof described in detail are exemplary embodiments that may be modified by the person skilled in the art in a wide variety of ways without departing from the invention. The use of the indefinite article “a” or “an” does not exclude the features concerned also being present multiple times. In addition, “units,” “devices,” “means,” and “elements” may include one or more components, also disposed spatially distributed.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An oral coil for a magnetic resonance tomography system for creating magnetic resonance images of a jaw area of a patient, the oral coil comprising:
a high-frequency antenna system including a plurality of high-frequency antennas; and
a shim system including a plurality of shim elements for homogenizing a static basic magnetic field of the magnetic resonance tomography system.

2. The oral coil as claimed in claim 1, wherein the high-frequency antenna system and the shim system form one constructional unit.

3. The oral coil as claimed in claim 1, wherein the oral coil, when used in or in front of the mouth of the patient, further comprises a curved housing for the high-frequency antenna system, the shim system, or the curved housing for the high-frequency antenna system and the shim system following a line of the jaw area.

4. The oral coil as claimed in claim 1, wherein the shim system comprises holders for shim elements of the plurality of shim elements.

5. The oral coil as claimed in claim 1, wherein at least one shim element of the plurality of shim elements comprises a passive element made of a material with defined magnetic susceptibility for adapting a B₀ field inhomogeneity.

6. The oral coil as claimed in claim 5, wherein the defined magnetic susceptibility for adapting the B₀ field inhomogeneity greater than 10².

7. The oral coil as claimed in claim 5, wherein the material comprises iron, cobalt, nickel or an alloy of iron, cobalt, and nickel.

8. The oral coil as claimed in claim 1, wherein at least one shim element of the plurality of shim elements comprises an electrical shim coil.

9. The oral coil as claimed in claim 8, wherein the shim system comprises a shim coil array including a plurality of electrical shim coils integrated into the shim system.

10. The oral coil as claimed in claim 1, wherein the high-frequency antenna system is disposed partly in an interoral housing part during operation.

11. The oral coil as claimed in claim 1, wherein the high-frequency antenna system is disposed in a housing part outside the head of the patient during operation.

12. The oral coil as claimed in claim 1, wherein shim elements of the plurality of shim elements are disposed in operation in a housing part outside the head of the patient.

13. A high-frequency receive device for a magnetic resonance tomography system for creation of magnetic resonance images of a jaw area, the high-frequency receive device comprising:
an oral coil comprising:
a high-frequency antenna system including a plurality of high-frequency antennas; and
a shim system including a plurality of shim elements for homogenizing a static basic magnetic field of the magnetic resonance tomography system.

14. The high-frequency receive device as claimed in claim 13, wherein the high-frequency antenna system and the shim system form one constructional unit.

15. The high-frequency receive device as claimed in claim 13, wherein the oral coil, when used in or in front of the mouth of the patient, further comprises a curved housing for the high-frequency antenna system, the shim system, or the curved housing for the high-frequency antenna system and the shim system following a line of the jaw area.

16. The high-frequency receive device as claimed in claim 13, wherein the shim system comprises holders for shim elements of the plurality of shim elements.

17. The high-frequency receive device as claimed in claim 13, wherein at least one shim element of the plurality of shim elements comprises a passive element made of a material with defined magnetic susceptibility for adapting a Bₒ field inhomogeneity.

18. A magnetic resonance tomography system for creating magnetic resonance images of a jaw area, the magnetic resonance tomography system comprising:
a head scanner including a basic field magnet; and
a high-frequency receive device comprising:
an oral coil comprising:
a high-frequency antenna system including a plurality of high-frequency antennas; and
a shim system including a plurality of shim elements for homogenizing a static basic magnetic field of the magnetic resonance tomography system.

19. A method for creating magnetic resonance images of a jaw area of a patient with an oral coil, the oral coil comprising a high-frequency antenna system and a shim system with a plurality of shim elements, the method comprising:
homogenizing a static basic magnetic field of a magnetic resonance tomography system with a head scanner for an acquisition of magnetic resonance signals using the high-frequency antenna system with the aid of the shim system.

20. A method of use comprising:
homogenizing a static basic magnetic field of a magnetic resonance tomography system for creating magnetic resonance images of a jaw area of a patient using an oral coil, the oral coil comprising a high-frequency antenna system and a shim system with a plurality of shim elements.
